SEQUENCE LISTING (31 sequences)

SEQ ID NO: 1 - DNA, 20 bp, Homo sapiens
ggctggtcttgaattcctca

SEQ ID NO: 2 - DNA, 20 bp, Homo sapiens
aggttgcagtgagccaagat

SEQ ID NO: 3 - DNA, 20 bp, Homo sapiens
gctcccaaccagacttttga

SEQ ID NO: 4 - DNA, 21 bp, Homo sapiens
gactgacaagagccacacaaa

SEQ ID NO: 5 - DNA, 20 bp, Homo sapiens
gttaccactcgcttccgatg

SEQ ID NO: 6 - DNA, 21 bp, Homo sapiens
cctcgttctcctgaatcagac

SEQ ID NO: 7 - DNA, 20 bp, Homo sapiens
catgtggagatcctgggttt

SEQ ID NO: 8 - DNA, 20 bp, Homo sapiens
gctgtggcaacagtatttgg

SEQ ID NO: 9 - DNA, 20 bp, Homo sapiens
cggaaggcatttctctgaac

SEQ ID NO: 10 - DNA, 20 bp, Homo sapiens
aagaaaccacaccagcaacc

SEQ ID NO: 11 - DNA, 20 bp, Homo sapiens
aggtgtgtcctgatgcttcc

SEQ ID NO: 12 - DNA, 20 bp, Homo sapiens
cctcactgaagccagagtgc

SEQ ID NO: 13 - DNA, 20 bp, Homo sapiens
gagtagaggcagtggcaggt

SEQ ID NO: 14 - DNA, 20 bp, Homo sapiens
cctccagtccttcaaagcat

SEQ ID NO: 15 - DNA, 19 bp, Homo sapiens
ttggctctttcgtcggact

SEQ ID NO: 16 - DNA, 20 bp, Homo sapiens
tccagcttagccttggtgat

SEQ ID NO: 17 - DNA, 20 bp, Homo sapiens
agtgcaacccaggctttcta

SEQ ID NO: 18 - DNA, 20 bp, Homo sapiens
tcacagagctgtggtcttgg

SEQ ID NO: 19 - DNA, 20 bp, Homo sapiens
ttgtgacacagaggagcctg

SEQ ID NO: 20 - DNA, 21 bp, Homo sapiens
cctcgttctcctgaatcagac

SEQ ID NO: 21 - DNA, 4193 bp, Homo sapiens
gtagatgaggaaactgaagtgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccccccatagggaaaccttttttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacctcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatatgcctgccttggcctctcaaagtgctgggattacaggcgtgagccactgtgcccggccttggctaacttttcaaaattaaagattttgacttgttacagtcatgtgacatttttttctttctgtttggtgagtttttgataatttatatctctcaaagtggagactttaaaaaagactcatctgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagagtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttattttaaaaaaaaaaaaaaaaaaaaaaaaaa

SEQ ID NO: 22 - protein, 1034 aa, Homo sapiens
MASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 23 - DNA, 20 bp, Homo sapiens
gtgaattctgcagctgttgg

SEQ ID NO: 24 - DNA, 20 bp, Homo sapiens
tgtaatccctgcactgagga

SEQ ID NO: 25 - DNA, 20 bp, Homo sapiens
ctcaactcctgcccagtgaa

SEQ ID NO: 26 - DNA, 19 bp, Homo sapiens
tgagctgagatcatgcact

SEQ ID NO: 27 - DNA, 23 bp, Homo sapiens
tcatttcacctccctaaattgaa

SEQ ID NO: 28 - DNA, 20 bp, Homo sapiens
ccctttggaaggaaattctg

SEQ ID NO: 29 - DNA, 23 bp, Homo sapiens
cccctggtatataaccccttaca

SEQ ID NO: 30 - DNA, 22 bp, Homo sapiens
cctgcctgataaagttgttttg

SEQ ID NO: 31 - DNA, 20 bp, Homo sapiens
ttgtgacacagaggagcctg

(19) United States
(12) Patent Application Publication
(10) Pub. No.: US 2004/0038224 A1
(43) Pub. Date: Feb. 26, 2004

(54) Isolated cryopyrins, nucleic acid molecules encoding these, and use thereof

(76) Inventors: Richard Kolodner, La Jolla, CA (US); Hal Hoffman, La Jolla, CA (US)

Correspondence Address: FULBRIGHT & JAWORSKI, LLP, 666 FIFTH AVE, NEW YORK, NY, 10103-3198, US

(21) Appl. No.: 10/264,958

(22) Filed: Oct. 4, 2002

Related U.S. Application Data
(60) Provisional application No. 60/327,728, filed on Oct. 5, 2001.

(51) Int. Cl. C12Q 1/68; C07H 21/04; C12N 9/02; C12P 21/02; C12N 5/06

(52) U.S. Cl. 435/6; 435/69.1; 435/189; 435/320.1; 435/325; 536/23.2

(57) ABSTRACT

The invention involves isolation of a new class of proteins, referred to hereafter as the cryopyrins. Also described are the isolated nucleic acid molecules involved in their expression. The inventive molecules are useful in diagnosing and treating inflammatory diseases, such as FCU/FCAS and MWS.

### RELATED APPLICATION

This application claims priority of provisional application Serial No. 60/327,728, filed Oct. 5, 2001, incorporated by reference in its entirety.

### FIELD OF THE INVENTION

This invention relates to nucleic acid molecules associated with familial cold urticaria, or familial cold autoinflammatory syndrome, as well as Muckle Wells Syndrome, the proteins encoded thereby, and uses thereof. Various aspects of the invention are disclosed.

### BACKGROUND AND PRIOR ART

Familial cold urticaria (“FCU” hereafter), was first described by Kile, et al., JAMA 114:1067-1068 (1940). It has been referred to as familial polymorphous cold eruption, cold hypersensitivity, cold pathergy and cold specific vasomotor neuropathy.

Clinical features of FCU include recurrent attacks of a nonpruritic, nonurticarial maculopapular exanthem associated with arthralgia, fever and chills, conjunctivitis, myalgia, headaches, fatigue and swelling of the extremities. See Tindall, et al., Arch. Intern Med 124:129-134 (1969); Zip, et al., Clin. Exp. Dermatol 18:338-341 (1993).

Onset of symptoms of FCU is generally delayed 30 minutes to 3 hours after exposure to cold, and these persist for 24-48 hours. The most consistent laboratory finding is marked, polymorphonuclear leukocytosis, which is found during attacks. See Tindall, et al., supra; Doeglas, et al., Arch. Dermatol 110:382-388 (1974).

Pathological changes which occur to the skin of patients during attacks include a primarily polymorphonuclear leukocyte perivascular infiltrate, increased vascularity, and dermal edema; however, no vasculitis is observed. See Martin, et al., Cutis 27:173-175 (1981); Tomesen, et al., Clin. Res. 33:690 (1985); Zip, et al., supra.

FCU manifests as early as birth, but generally not later than childhood. The condition persists through the subject's life. Generally, afflicted individuals have normal longevity, but some exhibit late-onset renal amyloidosis. Some patients also exhibit variants of Muckle-Wells syndrome, which is a condition that has variable expression, classically including recurrent rash, late-onset progressive nerve deafness, and late onset renal amyloidosis. See Muckle, et al., Br. J. Dermatol 100:87-92 (1979). Muckle-Wells syndrome patients exhibit phenotypes similar to FCU patients, but the symptoms are not precipitated by cold. See Muckle, supra; Jung, et al., Am. J. Hum. Genet. 59:A223 (1996).

With respect to inflammatory mediators, the only significant abnormalities in FCU patients are elevations of G-CSF and IL-6 (Urano, et al., Br. J. Dermatol 139:504-507 (1998)), and acute phase reactants such as C-reactive protein (Tonesen, et al., Aspen Allergy Conference Presentation, 1985).

Previously, Cuisset, et al., J. Hum. Genet 65:1054-1059 (1999), mapped Muckle-Wells Syndrome to chromosome 1q44. Hoffman, et al., Am. J. Hum. Genet 66:1693-1698 (2000), incorporated by reference, linked FCU to a locus on 1q44 as well. They describe a 10 cM region in which the gene is located.

None of these references describe the isolation and/or identification of a nucleic acid molecule associated with FCU or as it is also referred to, familial cold autoflammatory syndrome, or FCAS, or a nucleic acid molecule associated with Muckle-Wells Syndrome. An aspect of the invention is the isolation and identification of such nucleic acid molecules, and the ramifications thereof. Also disclosed are mutations within this gene which are associated with the disorder.

These will be elaborated upon in the detailed description which follows.

### BRIEF DESCRIPTION OF THE FIGURES

FIG. 1 set forth analysis of one family which includes individuals that suffer from FCAS.

FIG. 2 presents data on additional families which present heterozygous missense mutations.

FIG. 3 shows the structure of the CIAS 1 gene.

FIG. 4 details information on additional families analyzed for FCAS.

FIG. 5 presents haplotype analysis of selected individuals for the families analyzed.

FIGS. 6a-6d present variations on the physical map of the region relevant to the gene in question.

### DETAILED DESCRIPTION OF PREFERRED EMBODIMENTS

### EXAMPLE 1

Analysis of DNA was carried out on samples taken from members of 3 families which included at least two individuals who suffered from FCAS, and one family which included members suffering from MWS. The diagnosis of these individuals was based on a history of recurrent episodes of rash, arthralgia, and fever after generalized cold exposure. MWS subjects also suffered from sensoryneural hearing loss; however, the symptoms were not associated with exposure to cold.

Genomic DNA was isolated from peripheral blood samples taken from each subject. The DNA was then amplified, via PCR, and subjected to automated, fluorescent genotyping following Hoffman, et al., Am J. Hum Genet. 66:1693-1698 (2000), incorporated by reference, and microsatellite markers available in public databases.

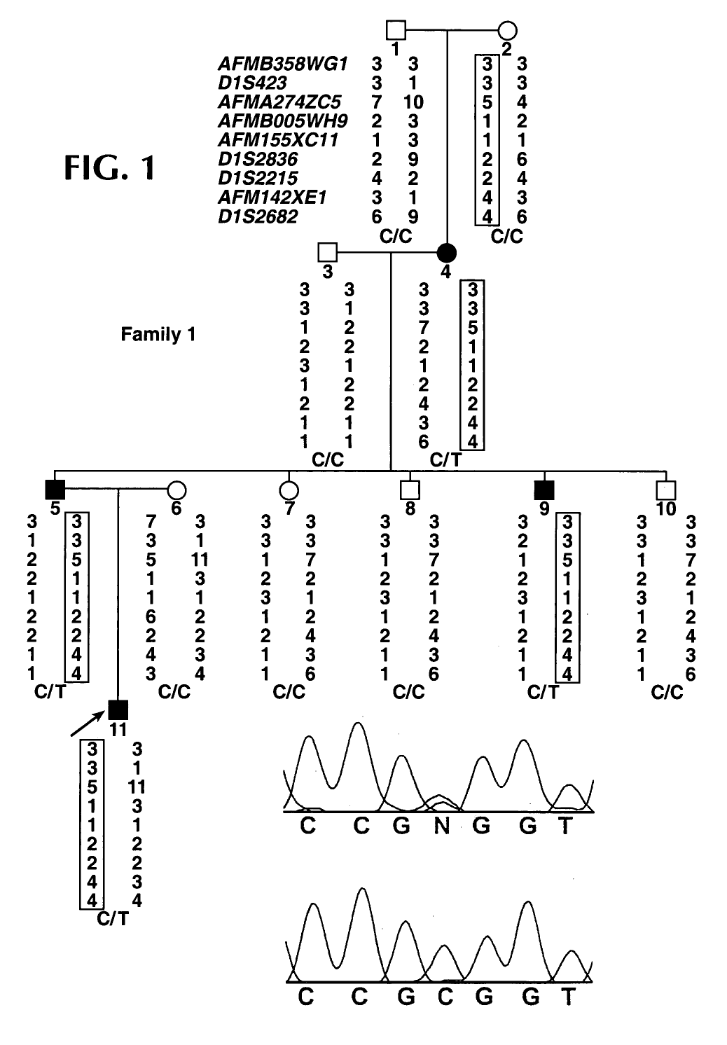

FIG. 1 presents the analysis of one such family. In this figure, the filled in figures represent individuals suffering from FCAS, while empty figures are unaffected individuals. The top of the figure indicates the microsatellite markers used, together with the allele numbers for each marker, as observed for each of the chromosome 1q44 haplotypes. Marker order was derived from several databases, and was confirmed by standard radiation hybrid mapping.

The boxed haplotype is the haplotype associated with FCAS. It is shared by all of the diseased individuals; however, note that it is also shared by a non-diseased individual. This suggests that a new mutation arose in individual “4,” which was also passed to descendants.

### EXAMPLE 2

These experiments describe further work on identifying and analyzing the coding region associated with FCAS and MWS.

Given the association of locus 1q44 with the disease, public data bases were screened to identify ESTs associated with this region. Once they were identified, these CDNA molecules were sequenced, assembled into longer sequence contigs to the extent possible, based upon overlap, extended, and then had their sequences confirmed, using a commercially available computer algorithm, i.e., “SEQUENCER 3.1.”

Primers were designed to be used in PCR amplification, based upon comparison of isolated cDNA sequences and the sequence of 1q44 in public data bases. The GENSCAN prediction program was used to analyze the publicly available information regarding 1q44. Some of the primers used to amplify regions of transcribed sequences in genomic DNA, were as follows:

| | |
|---|---|
| 5′-ggctggtcttgaattcctca-3′, | (SEQ ID NO: 1) |
| 5′-aggttgcagtgagccaagat-3′, | (SEQ ID NO: 2) |
| 5′-gctcccaaccagacttttga-3′, | (SEQ ID NO: 3) |
| 5′-gactgacaagagccacacaaa-3′, | (SEQ ID NO: 4) |
| 5′-gttaccactcgcttccgatg-3′, | (SEQ ID NO: 5) |
| 5′-cctcgttctcctgaatcagac-3′, | (SEQ ID NO: 6) |
| 5′-catgtggagatcctgggttt-3′, | (SEQ ID NO: 7) |
| 5′-gctgtggcaacagtatttgg-3′, | (SEQ ID NO: 8) |
| 5′-cggaaggcatttctctgaac-3′, | (SEQ ID NO: 9) |
| 5′-aagaaaccacaccagcaacc-3′, | (SEQ ID NO: 10) |
| 5′-aggtgtgtcctgatgcttcc-3′, | (SEQ ID NO: 11) |
| 5′-cctcactgaagccagagtgc-3′, | (SEQ ID NO: 12) |
| 5′-gagtagaggcagtggcaggt-3′, | (SEQ ID NO: 13) |
| 5′-cctccagtccttcaaagcat-3′, | (SEQ ID NO: 14) |
| 5′-ttggctctttctgtcggact-3′, | (SEQ ID NO: 15) |
| 5′-tccagcttagccttggtgat-3′, | (SEQ ID NO: 16) |
| 5′-agtgcaacccaggctttcta-3′, | (SEQ ID NO: 17) |
| 5′-tcacagagctgtggtcttgg-3′, | (SEQ ID NO: 18) |

These primers were designed to amplify exons with flanking intronic sequences. Amplification was carried out in 20 μl PCR reactions, using Taq DNA polymerase (0.8U), dNTPs (250 μM), MgCl₂ (2.5 mM), PE Buffer I (1×), primers (500 nM) and 20-80 ng of DNA taken from afflicted individuals, and a control who did not suffer from the syndrome, using commercially available reagents. The conditions used included initial denaturation at 94° C. for 4 minutes followed by 10 cycles at 94° C., 30 seconds per cycle, touchdown annealing (1° C. decrease per cycle) between 65-55° C., for 30 seconds, extension at 74° C. for 1 minute, and 25 additional cycles. The annealing temperature was 55° C., followed by a final extension at 72° C., for 7 minutes.

Ninety genomic fragments, containing more than 80 exons resulted, in total, using oligonucleotide primers in addition to those given, supra. These were purified, and sequenced via automated sequence techniques, in accordance with Kolodner, et al., Canc. Res. 59:5068-5074 (1999), incorporated by reference. Both the forward and reverse strands of PCR products were sequenced.

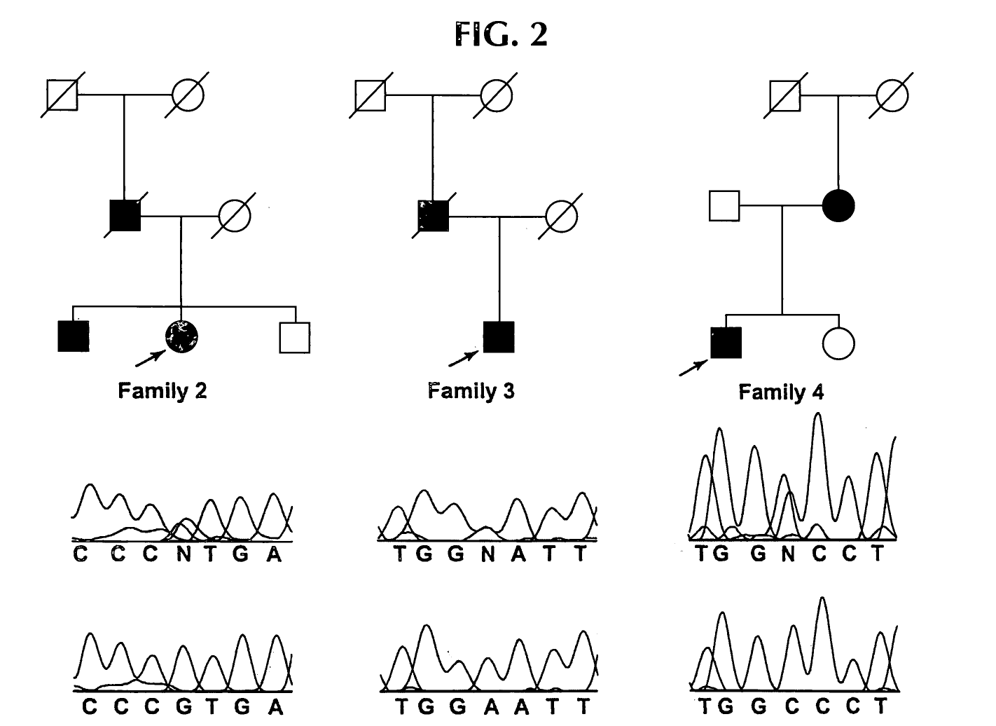

Sequencing of the amplified genonic DNA revealed 4 different missense mutations. Particulars of the position and specific mutations are presented infra. In terms of the individuals and families under analysis, the mutation which occurred in individual “4” appears to have arisen “de novo,” as only affected members of subsequent generations of the family inherited it, i.e., subjects 5, 9 and 11 in FIG. 1. Neither parent of subject “4” had the mutation, notwithstanding the presence of the disease haplotype in the mother. Three additional families, as shown in FIG. 2, possessed different, heterozygous missense mutations present in all afflicted subjects. One of the families had members diagnosed with Muckle-Wells Syndrome, and both suffered from sensorineural hearing loss. The family had a mutation in the same exon as the FCU/FCAS families. In a fourth family, individuals of which suffered from MWS, the phenotype appeared to arise as a result of a de novo mutation.

Following these results, controls were run on over 100 samples taken from unaffected family members, and taken from random North American blood banks. The mutations were not found in any of these samples. This absence supports the conclusion that the mutations described herein cause FCU/FCAS, and Muckle-Wells Syndrome.

The mutations referred to supra all appeared in the same exon, i.e., one consisting of 1753 base pairs. BLAST searching of public data bases identified two ESTs containing all or a part of this exon, i.e., AK 027194, and AF 054176. In all, analysis of publicly available genome sequences and sequences of BACs containing region 1q44 resulted in identification of 7 exons.

### EXAMPLE 3

The experiments described herein were designed to analyze the nucleic acid molecules more fully.

RT-PCR was carried out, via standard methods. Following sequencing, two additional exons and extensive alternative slice variants were identified, in the C-terminal region.

PCR products were obtained via amplifying human genomic DNA, chromosome 1q44, and RPI11 BAC clones (433k2, 978I15 AND 482N10), using standard methods, in order to characterize exons and flanking intron sequences.

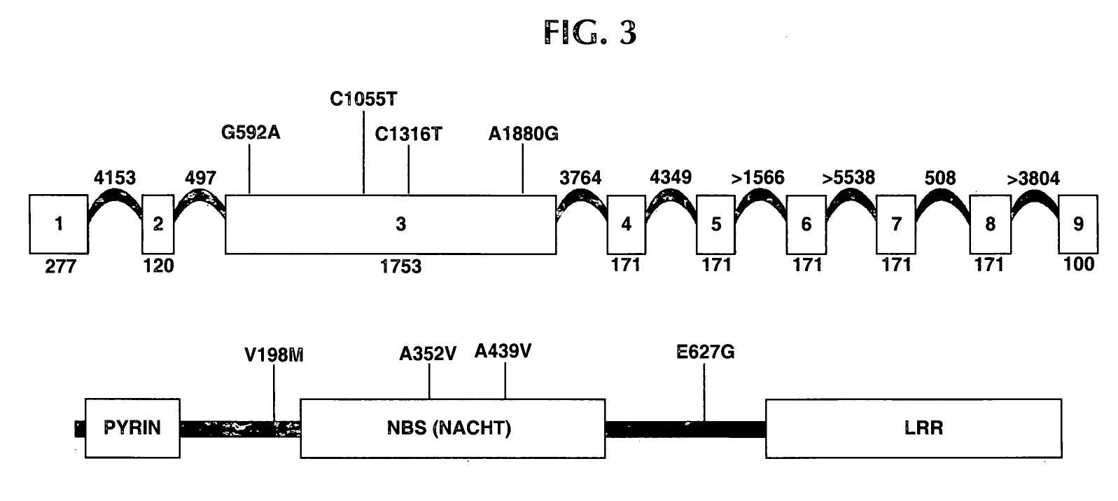

The experiments led to a predicted structure to the gene, as set forth in FIG. 3. The structure indicates that the gene contains 9 exons, encoding a 3105 base pair ORF. Two alternative start codons were found in the first exon, with the second one satisfying more Kozak criteria than the first. A stop codon was found in exon 9.

### EXAMPLE 4

These experiments describe Northern Blotting work. Probes were prepared corresponding to nucleotides-877 to 267, 1093 to 2150 and 2353-2970 of the ORF. A β actin control probe was also used. Probes were labeled with ^{α32}PdCTP, using commercially available materials and the instructions provided therein. Commercially available, multiple tissue blots were used.

The results identified a broad band of about 4 kb mRNA, expressed at low levels in peripheral blood lymphocytes, and with little or no expression in other tissue. This expression pattern is identical to that described for the “MEFV” gene, which is associated with Familial Medittaranean Fever. See Cell 90:797-807 (1997).

In experiments designed to verify the size of the mRNA, peripheral blood leukocyte mRNA was amplified, using primers designed from the genomic coding sequence, and the ends of the mRNA were amplified via RACE, using primers:

| | |
|---|---|
| ttgtgacaca gaggagcctg | (SEQ ID NO: 19) |
| and | |
| cctcgttctc ctgaatcagac, | (SEQ ID NO: 20) |

in accordance with the instructions from a commercially available product. The sequences were analyzed, and revealed that there was extensive alternative splicing of exons 4-8 of FIG. 3. The mRNA ranged in size from 3315 to 4170 base pairs. The pattern of distribution of the exons in the splice variants is set forth below:

1, 2, 3, 7, 8, 9-26.9%

1, 2, 3, 4, 5, 6, 7, 8, 9-16%

1, 2, 3, 5, 7, 8, 9-14.6%

1, 2, 3, 5, 6, 7, 8, 9-13.1%

1, 2, 3, 4, 5, 7, 8, 9-3.1%

1, 2, 3, 9-3.1%

1, 2, 3, 5*, 7, 8, 9-3.1%

1, 2, 3, 4, 7, 8, 9-2.4%

1, 2, 3, 5, 7, 9-1.5%

1, 2, 3, 8, 9-1.5%

1, 2, 3, 4, 9-1.5%

1, 2, 3, 4, 7, 9-0.8%

1, 2, 3, 6, 7, 8, 9-0.8%

1, 2, 3, 5, 8, 9-0.8%

1, 2, 3, 5, 6, 8, 9-0.8%

1, 2, 3, 4, 5, 6, 8, 9-0.8%

1, 2, 3, 4, 6, 7, 8, 9-0.8%

1, 2, 3, 4, 5*, 6, 7, 8, 9-0.8%

1, 2, 3, 5*, 6, 7, 8, 9-0.8%

1, 2, 3, 5, 6, 7**, 8, 9-0.8%

with reference to the open reading frame, these exons are encoded by nucleotides in parenthesis:

1 (1-277), 2 (278-397), 3 (298-2150), 4 (2151-2321), 5 (2322-2492), 6 (2493-2663), 7 (2664-2834), 8 (2835-3005) and 9 (3006-3105).

5* is a splicing event where the upstream exon is spliced to a site 57 bp downstream of exon 5. 7** is a splicing event where the upstream exon is spliced to a site 140 bp upstream of exon 7, resulting in an in frame stop codon downstream of the splice junction.

### EXAMPLE 5

One of the splice forms identified in the examples described supra is set forth at SEQ ID NO: 21. It contains exons 1-3, 5, 7 and 9. The deduced amino acid sequence (SEQ ID NO.: 22) consists of 920 amino acids, a size of about 105.7 kilodaltons as determined by SDS-PAGE, and a pI of about 6.16. Additional sequence information is also provided.

The sequence contains several distinct motifs. Specifically, a pyrin domain is found at amino acids 13-83 (see Martinin, et al., Curr. Biol 11:R118-R120 (2001); Bertin, et al, Cell Death Differ 7:1273-1274 (2000); Pawlowski, et al., Trends Biochem Sci 26:85-97 (2001)), a central NBS from the NACHT subfamily (Koonin, et al., Trends Biochem Sci 25:223-224 (2000)), in exon 3 at amino acids 217-533, and a C-terminal LRR domain, with 7 LRRs contained within amino acids 697-920. “LRRs” are known to the skilled artisan as domains of proteins which bind to other proteins. See Kobe, et al., Nature 374:183-186 (1995). No nuclear localization signals were identified, nor were any clear transmembrane regions found.

If all 9 exons are transcribed, the largest protein produced contains 1034 amino acids, is about 117.8 kD in size, and contains 11 LRRs. These features are consistent with the protein being a signaling protein, involved in regulation of inflammation and apoptosis. The proteins will be referred to collectively as cropyrins, and their encoding molecules as forms of the CIAS1, (cold induced autoinflammatory syndrome 1) gene.

### EXAMPLE 6

In further experiments, informed consent was obtained, and a total of 179 individuals, from four families which included sufferers from FCAS, were analyzed. Of these 179 individuals, 105 were unaffected, and 74 were affected.

Pedigree information and family history information were obtained from multiple sources within each family. Detailed information on pedigrees can be found at Hoffman, et al, J. Allergy Clin. Immunol 108:615-620 (2001), incorporated by reference. The individual names and pedigree structures were evaluated extensively for relatedness between families. No relationship was ascertained.

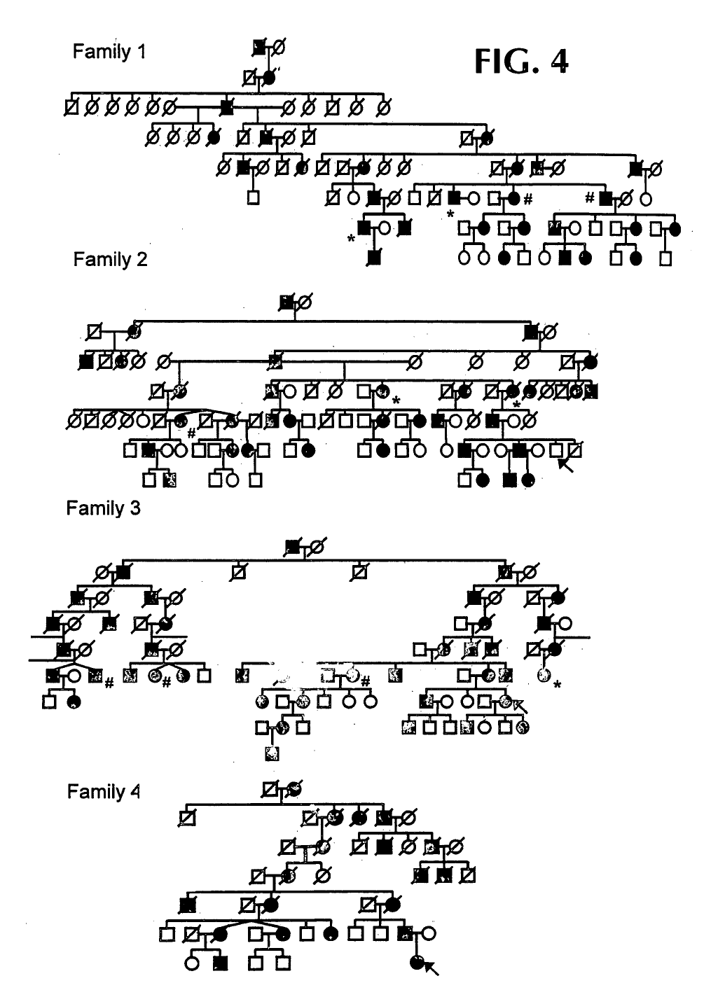

Representative segments of the pedigrees are presented in FIG. 4. Filled squares represent afflicted males, and filled circles afflicted females. Unaffected individuals are represented by open figures.

The pedigrees demonstrate an autosomal dominant inheritance pattern, with near complete, penetrance, and a transmission rate of 50%. The earliest affected ancestors lived in the 17^{th} century.

As noted, supra, there was no evidence of relatedness between families based upon names or pedigree structure; however, members of the first three families share a legend concerning the origin of the disorder, relating to a healthy man who had a prolonged, life threatening exposure to cold water. Following the exposure, the individual developed symptoms of FCAS, and passed them onto offspring.

### EXAMPLE 7

Following the results discussed supra, blood samples were taken from 179 members of the four families described above. Of these, 74 were afflicted individuals, and 105 were unafflicted. Standard venipuncture and genomic DNA isolation techniques were used.

Clones from the FCAS locus were identified, using oligonucleotides derived from genetic markers that had been marked previously. These oligonucleotides were labeled with ³²P, and were hybridized to RPCI-11BAC, in accordance with Shizuya, et al, Proc. Natl. Acad. Sci. USA 89:8794-8797 (1992), and RPCI-4PAC, in accordance with Ioanormu, et al, Nat. Genet. 6:84-89 (1994).

Further oligonucleotides were designed, using clone end sequences, labeled with ³²P, and then hybridized to filters, in order to identify overlapping clones, via chromosome walking.

A group of 31 clones, covering over 60% of the region, were sent to Wellcome Trust Sanger Institute for incorporation into their chromosome 1 physical map.

Restriction digest fingerprints of the clones were prepared, in accordance with Gregory, et al, Genome Res. 7:1162-1168 (1997), incorporated by reference, and these were compared to chromosome 1 data set using fingerprint contigs, in accordance with Soderland, et al, Comput. Appl. Biosci. 13:523-535 (1997), incorporated by reference. When fingerprint overlaps were found to be statistically significant, they were assimilated into the map.

Two clones formed the basis of the sequence tiling map, discussed infra, and one facilitated the cloning of a gap in the map of chromosome 1.

### EXAMPLE 8

All 179 subjects were then genotyped in the 10 cM region between markers D1S423 and D1S2682, in order to identify rare cross over events. Ten of the oldest affected and available family members from these families were also genotyped in the 50 cM region between markers D1S549 and D1S2682, in order to evaluate shared haplotypes. Microsatellite markers were identified in a series of public databases, including www.chlc.org; www.genethon.fr; www.cedargenetics.soton.ac.uk; www.gdb.org; www.morshmed.org/genetics; and www.genome.wi.mit.edu. Additional microsatellite markers were designed by searching the public genome database in the region (www.ncbi.nlm.nih.gov/entrez), for short, tandem repeat sequences greater than 20 base pairs long. Flanking oligonucleotides primers were designed, and PCR was carried out as described by Hoffman, et al, Am. J. Hum. Genet. 66:1693-1698 (2000), incorporated by reference. Haplotypes were constructed using ordered genotypes from multiple family members, in order to identify recombination events and to recognize shared alleles, using the commercially available program Cyrillic 3.1.

The primer sequences designed and used for markers were:

| | |
|---|---|
| gtgaattctg cagctgttgg | (SEQ ID NO: 23) |
| and | |
| tgtaatccct gcactgagga | (SEQ ID NO: 24) |
| for D1S3770; | |
| ctcaactcct gcccagtgaa | (SEQ ID NO: 25) |
| and | |
| tgagctgaga tcatgcact | (SEQ ID NO: 26) |
| for D1S3771; | |
| tcatttcacc tccctaaatt gaa | (SEQ ID NO: 27) |
| and | |
| ccctttggaa ggaaattctg | (SEQ ID NO: 28) |
| for D1S3772; and, | |
| cccctggtat ataacccctt aca, | (SEQ ID NO: 29) |
| and | |
| cctgcctgat aaagttgttt tg | (SEQ ID NO: 30) |
| for D153773. | |

These four markers, together with known markers, were used to study haplotypes. The results, set forth in Table 1, which follows, show that families 1-3 share a pattern of multiple alleles for polymorphic microsatellite markers at 1q44, indicating a common serial haplotype of approximately 10 cM between D1S423 and D1S2682. In this table, critical mapped regions are shaded. “MB” stands for the USCS Golden Path genomic map, while “cM” is based upon the Genethon genetic map.

TABLE 1
Microsatellite markers in the FCAS locu

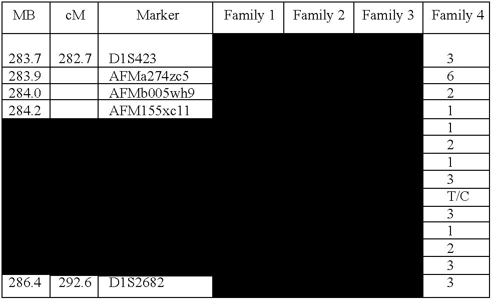

MB - based on UCSC Golden Path genomic map;
cM - based on Genothon genetic map
Lightly shaded region indicates critical mapped region
Darker shaded region indicates shared haplotype

Several individuals, marked by asterisks in FIG. 4, were genotyped over a 50 cM region. These individuals are marked by a thatch (“#” mark). They shared a much longer haplotype of approximately 40 cM, between D1S3462, and D1S2682.

Further pedigree analysis and family history did not uncover a common ancestor, suggesting the familial connection must extend beyond current data, to at least 8 generations in the past. Family 4 was not seen to share the hapoltype.

### EXAMPLE 9

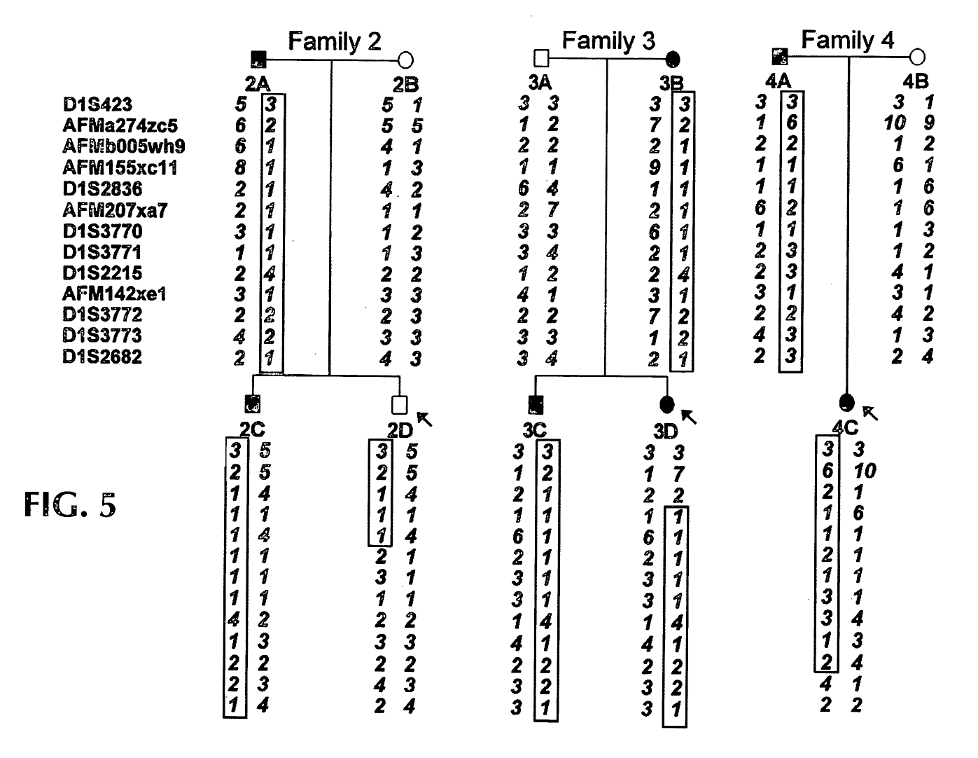

Haplotype analysis was carried out on all 4 families, as described, supra. Informative recombination information was found in some individuals, and this information is set forth in FIG. 5. Ordered STS markers were used, and are presented to the left of subject 2A. The allele numbers for each microsatellite marker are presented for the 2 chromosomes 1q44 haplotypes which were inherited by each individual. FIG. 2 shows the haplotype associated with FCAS, boxed.

There were key, crossover events found in unaffected patient 2D, where there was a crossover between D1S2836 and AFM 207xa7, one in affected patient 3D, between AFMb005wh9 and AFM155xc11, and one in affected patient 4C, between D1S3772 and D1S3773. Recombinational analysis of all of the family data suggests a centromeric limit at D1S2836 and a telomeric limit at D1S3773.

### EXAMPLE 10

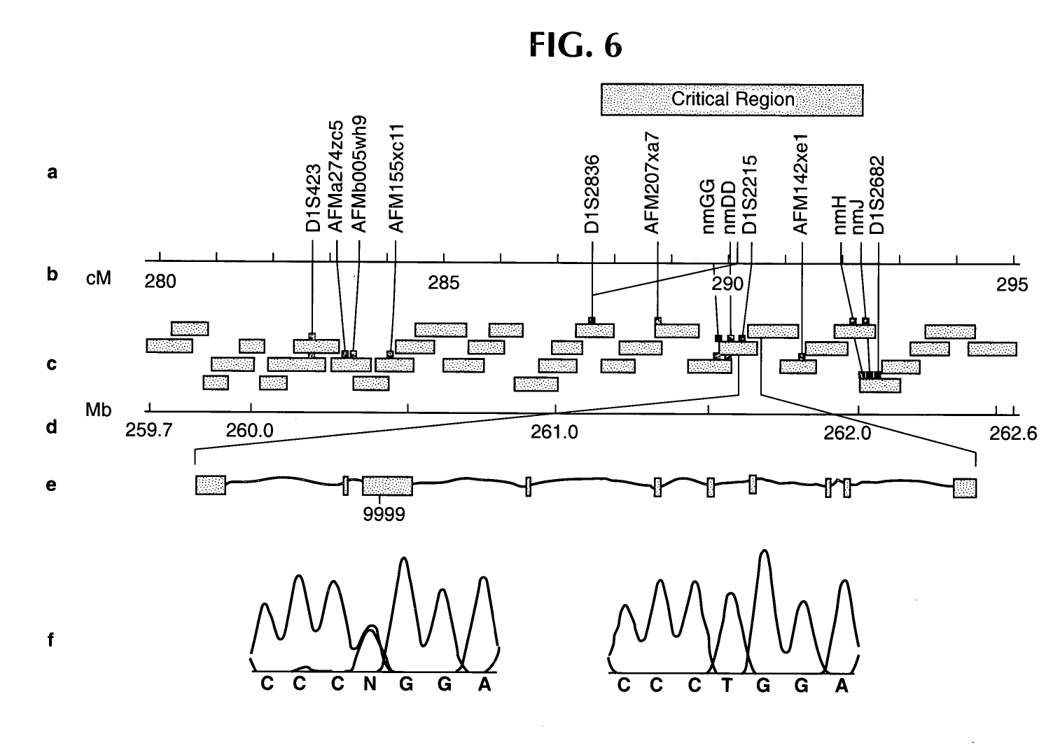

A complete physical map of the region extending from D1S423 through D1S2682 was constructed, using standard chromosomal working and BAC fingerprinting methods. This map is set forth in FIGS. 6a-6d. It shows the filing of 34 BACs covering the region. FIG. 6a shows the location of 13 ordered microsatellite markers on the Genethon genetic map, which can be found at www.genethon.fr, and is incorporated by reference. This covers about 10 cM. FIG. 6C shows corresponding BACs, while FIG. 6d shows a map, based upon USC Genome Bioinformatics (www.genome.USC.edu), which covers about 3 Mb. The critical region, i.e., that discussed supra, between D1S8236 and D1S3773, in less than 1 Mb in size.

### EXAMPLE 11

Examples 1-5, supra, describe the isolation and identification of the CIAS1 gene. An intron/exon map is set forth at FIG. 6e. Exons 1-6 are found on BAC-RPCI-11 433K2, while exons 7-9 are found on BAC-RPCI-11 97815.

### EXAMPLE 12

This example describes experiments which were carried out to determine if any additional mutations within the CIAS1 gene were connected to FCAS.

The primers set forth in example 2 were used, as was an additional primer based upon exon 3, i.e.:

ttgtgacaca gaggagcctg (SEQ ID NO: 31).

PCR was carried out as described in example 2, as was purification and sequencing. Normal controls were unaffected spouses, over 100 normal blood bank control DNA samples, and 50 DNA samples of rheumatoid arthritis patients. Allele frequencies were calculated by dividing the number of base changes found, by the total number of chromosome sequenced, which was about 400.

A total of 11 single nucleotide polymorphisims, or “SNPs” were found, as shown in Table 2. Two are rare (less than 1%) variations, and all but one were located in exon 3. All disease causing mutations were found in exon 3. With the exception of a change at nucleotide 2107, all of these mutations are silent. Three of the mutations, i.e., G780A, C930T, and C1302T, are located within a few base pairs of previously identified disease causing mutations, i.e., C778T, T926C, C1307A. See Dode, et al, Am. J. Hum. Genet. 71:198-203 (2002). Dode, et al, had reported four of these SNPs previously, with similar allele frequency, in a European population.

TABLE 2
Single nucleotide polymorphisims within CIAS1

| | Amino | | Frequency | | Frequency |
|---|---|---|---|---|---|
| Nucleotide | Acid | Allele | (%) | Allele | (%)* |
| 657 | 219 | C | 92.4 | T | 7.6 |
| 726 | 242 | A | 56.0 | G | 44.0 |
| 780# | 260 | G | 97.7 | A | 2.3 |
| 930# | 310 | C | 99.5 | T | 0.5 |
| 1231 | 411 | C | 98.5 | T | 1.5 |
| 1302# | 434 | C | 84.5 | T | 15.5 |
| 1383 | 461 | C | 99.0 | T | 1.0 |
| 1389 | 463 | C | 99.5 | T | 0.5 |
| 1600 | 534 | C | 99.7 | T | 0.3 |
| 2107 | Q703K | C | 97.0 | A | 3.0 |
| Intron + 59 | — | G | 96.5 | A | 3.5 |

*allele frequency based on 400 chromosomes tested
#indicates SNPs in close proximity to disease causing mutations

The foregoing examples set forth variants features of the invention, including various members of the cryopyrin protein family and nucleic acid molecules which encode them. “Cryopyrin” as used herein, refers to any and all forms of the protein described herein. As used herein “cryopyrin” refers to any protein which comprises, in its amino acid sequence, at least the amino acids encoded by exons 1, 2 and 3 of the CISA1 gene, as described herein. In the alternative, with reference to the open reading frame, these proteins include amino acids encoded by nucleotides 2150, in SEQ ID NO: 21. Additional embodiments relate to proteins which are encoded by nucleotide sequences which comprise nucleotides 1-2150, followed by additional nucleotides, and ending with nucleotides 3006-3105. An especially preferred embodiment is a protein encoded by nucleotides 1-2150, concatenated to nucleotides 2834-3105 of SEQ ID NO: 21. The various splice variants described herein, together with the portions of the ORF which encode them, are described supra. All of these, as well as the encoded proteins, expression vectors which comprise the nucleic acid molecule splice variants operably linked to a promoter, and cells transformed or transfected thereby, are part of the invention.

While human molecules are described, other species are included in the invention, such as mammalian molecules. Exemplary, but by no means exclusive examples of such molecules include murine, bovine, and other species. Especially preferred are molecules which exhibit at least 60%, preferably at least 70%, and most preferably, at least 80% homology with the human splice variants described herein.

It will be understood by the skilled artisan that the splice variants described above are not limiting, and other splice variants should be expected. While it can perhaps be assumed that most of these will include exons 1, 2, and 3, this is not a requirement, and the inventors do not wish to be bound to such a requirement.

Similarly, nucleic acid molecules which encode the missense forms described herein are a feature of the invention, as are the resulting mutants. As pointed out, supra, mutations associated with various positions, such as 592, 657, 726, 780, 930, 1055, 1231, 1302, 1316, 1383, 1389, 1600, 1880 and 2107 of the open reading frame are in turn associated with FCU/FCAS and MWS. Hence, any variant nucleic acid molecule and/or protein with a mutation relative to the wild type molecules is a feature of this invention, including the specific mutations described herein.

Also a feature of this invention are methods for diagnosing disorders. As has been shown, supra, missense and silent mutations within the CIAS1 gene are associated with disorders such as FCU/FCAS and MWS. One of ordinary skill in the art can envision other disorders, associated with the mutations described herein, or others in the CIAS1 gene. Various methodologies for identifying mutations in genes, transcripts and proteins, are all well known to the art and need not be elaborated upon in detail. Exemplary of such assays are hybridization assays, such as assays based upon labelled oligonucleotide probes, PCR, other assays based upon nucleic acid amplification, assays based upon enzymatic cleavage with restriction endonucleases, and so forth. Proteins based assays, such as immunoassays, electrophoretic assays, and so forth, are also features of the invention.

As noted, supra, the mutations in the proteins and nucleic acid molecules are associated with FCU/FCAS and MWS, while the wild type proteins are not. Both FCU/FCAS and MWS are inflammation related disorders. Hence, a further aspect of the invention is the treatment of inflammatory disorders, including FCU/FCAS and MWS, via administration of appropriate forms of wild type protein. Such forms of wild type protein include an amino acid sequence including at least the wild type amino acids corresponding to the relevant mutation or mutations. These can be determined via standard methodologies for determining the nucleotide sequence of the gene from the afflicted individuals. The proteins may be administered in any of the standard methods used for administration of proteins, such as intravenous administration, subcutaneous administration, etc. The proteins can be combined with various substances to optimize their delivery and/or efficacy, such as standard pharmaceutical carriers. They can be formulated for timed or delayed release, etc.

The multiple splice patterns described supra include molecules with varying numbers of LRRs. As noted, supra, LRRs are motifs involved in protein-protein interaction and binding. Differences in LRR content suggest that the different proteins interact with a variety of other molecules, and such interactions may be involved in the development or prevention of pathological processes, including inflammation. One aspect of the invention is a method for identifying molecules which interact with one or more of the splice variants of the invention, by contacting the molecule of interest with a splice variant, and determining binding there between. One can identify those molecules which bind specifically to one or less than all variants, by screening against a plurality of variants. Such a method is especially useful in situations where, e.g., a variant is expressed in particular tissue types, at development stages, in connection with particular disorders, etc. It has also been found that when CIAS1 patients are heat induced, extraordinarily high levels of interleukin-6 (“IL-6”) are released, suggesting a therapeutic role for this molecule in treatment of the disorders set forth herein.

Further features of the invention include antibodies, such as monoclonal antibodies, humanized antibodies, fragments of antibodies which bind to the proteins of the invention, hybridimas which produce the antibodies, and so forth are also a part of the invention.

Other features of the invention will be clear to the artisan and need not be set forth herein.

We claim:

1. An isolated protein comprising the amino acid sequence of wild type cryopyrin as set forth in SEQ ID NO: 22, with the proviso that (i) amino acid 198 is not Val (ii) amino acid 352 is not Ala, (iii) amino acid 434 is not Ala, (iv) amino acid 627 is not Glu, or (v) amino acid 703 is not Gln.

2. The isolated protein of claim 1, wherein amino acid 198 is Met rather than Val.

3. The isolated protein of claim 1, wherein amino acid 352 is Val rather than Ala.

4. The isolated protein of claim 1, wherein amino acid 439 is Val rather than Ala.

5. The isolated protein of claim 1, wherein amino acid 627 is Gly rather than Glu.

6. The isolated protein of claim 1, wherein amino acid 703 is Lys rather than Gln.

7. An isolated nucleic acid molecule which encodes the protein of claim 1.

8. An isolated nucleic acid molecule which encodes the protein of claim 2.

9. An isolated nucleic acid molecule which encodes the protein of claim 3.

10. An isolated nucleic acid molecule which encodes the protein of claim 4.

11. An isolated nucleic acid molecule which encodes the protein of claim 5.

12. An isolated nucleic acid molecule which encodes the protein of claim 6.

13. Expression vector comprising the isolated nucleic acid molecule of claim 7, operably linked to a promoter.

14. Recombinant cell comprising the isolated nucleic acid molecule of claim 7.

15. Recombinant cell comprising the expression vector of claim 13.

16. A method for determining presence of a disorder comprising assaying a sample taken from a subject believed to suffer from said disorder for a mutation in the nucleic acid molecule which encodes cryopyrin encoded by SEQ ID NO: 22, presence of said mutation being indicative of possible presence of said disorder.

17. The method of claim 16, said method comprising polymerase chain reaction.

18. The method of claim 16, wherein said mutation is a mutation at the codon which encodes amino acid 198 of cryopyrin.

19. The method of claim 16, wherein said mutation is a mutation at the codon which encodes amino acid 352 of cryopyrin.

20. The method of claim 16, wherein said mutation is a mutation at the codon which encodes amino acid 439 of cryopyrin.

21. The method of claim 16, wherein said mutation is a mutation at the codon which encodes amino acid 627 of cryopyrin.

22. The method of claim 16, wherein said mutation is a mutation at the codon which encodes amino acid 703 of cryopyrin.

23. The method of claim 16, wherein said mutation occurs in both alleles of said subject's gene which encodes cryopyrin.

24. The method of claim 23, wherein both of said alleles carry the same mutation

25. The method of claim 23, wherein each of said alleles carries a different mutation.

26. An isolated polypeptide comprising amino acids 13-83 of SEQ ID NO: 22, amino acids 217-533 of SEQ ID NO: 22, or amino acids 697-920 of SEQ ID NO: 22

27. The isolated polypeptide of claim 26, comprising all of amino acids 13-83, 217-533, and 697-920 of SEQ ID NO: 22.

28. The isolated polypeptide of claim 26, comprising the amino acid sequence of SEQ ID NO: 22, or a portion thereof.

29. An isolated polypeptide comprising amino acids 1-83 of SEQ ID NO: 22.

30. The isolated polypeptide of claim 28, comprising amino acids 1-533 of SEQ ID NO: 22.

31. The isolated polypeptide of claim 28, comprising amino acids 1-533 of SEQ ID NO: 22.

32. The isolated polypeptide of claim 28, comprising amino acids 1-920 of SEQ ID NO: 22.

33. An isolated nucleic acid molecule which encodes the isolated polypeptide of claim 28.

34. The isolated nucleic acid molecule of claim 33, wherein said isolated nucleic acid molecule is cDNA.

35. The isolated nucleic acid molecule of claim 33, wherein said isolated nucleic acid molecule is genomic DNA.

36. The isolated nucleic acid molecule of claim 33, comprising the nucleotide sequence of SEQ ID NO: 21.

37. An antibody which binds specifically to the polypeptide of claim 26.

38. An isolated antibody which binds specifically to the protein of claim 1.

39. An isolated nucleic acid molecule which encodes a cryopyrin, the nucleotide sequence of which comprises nucleotides 1-2150 of SEQ ID NO: 21.

40. An isolated protein encoded by the isolated nucleic acid molecule of claim 38.

41. The isolated nucleic acid molecule of claim 40, further comprising nucleotides 3006-3105 of SEQ ID NO: 21, positioned downstream of nucleotides 1-2150 of SEQ ID NO: 21.

42. An isolated protein encoded by the isolated nucleic acid molecule of claim 41.

43. A method for treating inflammation, comprising administering to a patient suffering from an inflammation an amount of the isolated protein of claim 39 sufficient to alleviate said inflammation.

44. The method of claim 42, wherein said patient suffers from FCU/FCAS or MWS.

45. A method for identifying a substance useful in modulating binding of a cryopyrin protein to a second protein, comprising admixing said substance and a cryopyrin protein, and determining binding of said substance to said cryopyrin, wherein said binding is indicative of a substance useful in modulating binding of said cryopyrin to another protein.

46. An isolated nucleic acid molecule which encodes the encryopyrin protein that is encoded by the nucleotide sequence of SEQ ID NO: 21, with the provisio that said isolated nucleic acid molecule contains a silent mutation at least one of the following nucleotides: nucleotide 657, 726, 780, 930, 1231, 1302, 1383, 1389, or 1600.

47. An isolated oligonucleotide useful in diagnosing a disorder characterized by an aberrant CIAS1 gene, wherein said oligonucleotide comprises from about 10 to about 100 nucleotides, and contains at least one of the mutations of the isolated nucleic acid molecule of claim 46.
